(19) United States
(12) Patent Application Publication
(10) Pub. No.: US 2020/0024296 A1
(43) Pub. Date: Jan. 23, 2020

(54) PROCESS FOR THE ISOLATION OF NOVEL GLYCOSIDES FROM PTEROCARPUS MARSUPIUM AND THEIR THERAPEUTIC EFFECTS

(71) Applicants: Muhammed Majeed, Bangalore (IN); Kalyanam Nagabhushanam, East Windsor, NJ (US)

(72) Inventors: Muhammed Majeed, Bangalore (IN); Kalyanam Nagabhushanam, East Windsor, NJ (US)

(21) Appl. No.: 16/515,298

(22) Filed: Jul. 18, 2019

Related U.S. Application Data
(60) Provisional application No. 62/700,446, filed on Jul. 19, 2018.

(51) Int. Cl. C07H 7/06; C07H 1/08; A61K 36/48; A61P 3/08

(52) U.S. Cl. CPC C07H 7/06; A61P 3/08; A61K 36/48; C07H 1/08

(57) ABSTRACT

The present invention discloses a composition comprising not less than 5% w/w Pterocarpus marsupium extract standardized to contain not less than 0.5% w/w Pterocarposide and not less than 0.5% w/w Sabioside and a method for isolating the same. The invention also discloses a method of activating AMPK and inhibiting glucose production in mammalian cells using the aforementioned composition.

### CROSS-REFERENCE TO RELATED PATENT APPLICATIONS

The invention is non-provisional filing of U.S. provisional patent application No. 62/700,446 filed on 19 Jul. 2018.

### BACKGROUND OF THE INVENTION

### Field of the Invention

The invention in general relates to active molecules from Pterocarpus marsupium. More specifically, the present invention relates to a process for the isolation of novel C-glycosides from Pterocarpus marsupium and their therapeutic effects thereof.

### Description of Prior Art

Pterocarpus marsupium is a deciduous tree that is native to the parts of India, Nepal and Sri Lanka. It contains many flavonoids, glycosides, catechins, stilbenoids and tannins that exhibit therapeutic properties. Pterocarpus marsupium is reported to have a positive effect in the management of diarrhea, toothache, fever, urinary tract and skin infections. (S. S. Handa et al., Pterocarposide, an isoaurone C-glycoside from Pterocarpus marsupium, Tetrahedron Letters 41 (2000) 1579-1581). The C-glycosides isolated from Pterocarpus marsupium are reported to possess anti-hyperglycemic activity. However, most of the C-glycosides from the plant remain to be identified to completely tap its therapeutic potential.

Adeosine Mono Phosphate-activated protein kinase (AMPK) has been known for many years as a central metabolic regulator to inhibit energy-consuming pathways as well as to activate the compensating energy-producing programs. The AMPK (enzyme is activated when there are changes in the cellular energy status such as muscle contraction and hypoxia. AMPK can be pharmacologically activated by the compound 5-aminoimidazole-4-carboxamide ribonucleoside (AICAR) and the anti-diabetic agent Metformin. AMPK plays an important role in the stimulation of muscle glucose uptake by these physiological and pharmacological stimuli. Activation of AMPK during myocardial ischemia both increases glucose uptake and glycolysis while also increasing fatty acid oxidation during reperfusion. The following articles disclose the role of activation of AMPK.
1. Ha J, Guan K L, Kim J, AMPK and autophagy in glucose/glycogen metabolism, Mol Aspects Med. 2015 December; 46:46-62
2. N. Musi and L. J. Goodyear, AMP-activated protein kinase and muscle glucose uptake, Acta Physiol Scand 2003, 178, 337-345.
3. Sambandam N, Lopaschuk G D, AMP-activated protein kinase (AMPK) control of fatty acid and glucose metabolism in the ischemic heart, Prog Lipid Res. 2003 May; 42(3):238-56

AMPK is now a therapeutic target for managing metabolic disorders likes diabetes, obesity etc. Inhibiting gluconeogenesis is also important for reducing the production of ketone bodies in people with diabetes, which can otherwise prove detrimental (Blackshear et al., The effects of inhibition of gluconeogenesis on ketogenesis in starved and diabetic rats, Biochemical Journal 1975, 148 (3): 353-362).

Previous studies have been successful in identifying the flavonoids and glycosides from Pterocarpus marsupium (Bezuidenhoudt et al., Flavonoid Analogues from Pterocarpus Species Phytochemistry. Vol. 26. No 2. Pp. 531-535. 1987), but were unable to isolate some of the C-glycosides in their pure form to elucidate their biological activity. The present invention discloses a process for identifying novel C-glycosides from Pterocarpus marsupium and their therapeutic effect.

The principle object of the invention is to disclose a process for the isolation of C-glycosides—Pterocarposide (CAS no. 264876-26-8) and Sabioside (CAS no. 108351-24-2) from Pterocarpus marsupium.

It is another objective of the invention to disclose a composition comprising C-glycosides Pterocarposide (STR#1) and Sabioside (STR#2) isolated from Pterocarpus marsupium and its therapeutic potential in activating AMPK and inhibiting gluconeogenesis.

The present invention solves the above mentioned objectives and provides related advantages.

### SUMMARY OF THE INVENTION

In a preferred embodiment, the present invention discloses a process for the isolation of C-glycosides Pterocarposide (STR#1) and Sabioside (STR#2) from Pterocarpus marsupium.

In a related embodiment, the invention discloses a composition comprising not less than 5% w/w Pterocarpus marsupium extract standardized to contain not less than 0.5% w/w Pterocarposide (STR#1) and not less than 0.5% w/w Sabioside (STR#2).

In another preferred embodiment, the invention discloses a method of activating AMPK in mammalian cells, comprising step of bringing into contact mammalian cells with a composition comprising not less than 5% w/w Pterocarpus marsupium extract standardized to contain not less than 0.5% w/w Pterocarposide (STR#1) and not less than 0.5% w/w Sabioside (STR#2), to bring about the effect of AMPK activation.

In another preferred embodiment, the invention discloses a method of inhibiting gluconeogenesis in mammalian cells, said method comprising steps of bringing into contact mammalian cells with a composition comprising not less than 5% w/w Pterocarpus marsupium extract standardized to contain not less than 0.5% w/w Pterocarposide (STR#1) and not less than 0.5% w/w Sabioside (STR#2), to bring about the effect of reduction in glucose production.

Other features and advantages of the present invention will become apparent from the following more detailed description, taken in conjunction with the accompanying images, which illustrate, by way of example, the principle of the invention.

### BRIEF DESCRIPTION OF THE FIGURES

FIG. 1 is a western blot image showing the activation of AMPK in H4IIE cells by the Pterocarposide composition.

FIG. 2 is a graphical representation showing the increase in the expression of pAMPK in HepG2 cells by the Pterocarposide composition.

FIG. 3 is a graphical representation showing the decrease in glucose production in H4IIE cells by the Pterocarposide composition.

### DESCRIPTION OF THE MOST PREFERRED EMBODIMENTS

In a most preferred embodiment, the invention discloses a process for isolating C-glycosides from Pterocarpus marsupium, said process comprising steps of:
a) Charging Pterocarpus marsupium wood powder into an extractor
b) Extracting with a solvent to obtain an oleoresin
c) Dissolving the oleoresin of step b) in water and extracting with a solvent to obtain a aqueous layer and solvent layer
d) Further washing the solvent layer of step c) with water to further obtain an aqueous and solvent fractions
e) Mixing the aqueous fractions of step c) and step d) and spray drying to obtain an aqueous extract
f) Fractionating the aqueous fraction of step e) with a solvent to obtain an aqueous fraction and solvent fraction
g) Passing the solvent fraction of step f) through a solvent gradient column by using Silica gel 60-120 mesh to obtain enriched fraction 1 and fraction 2
h) Passing the enriched fraction 1 from step g) through isocratic RP-18 Silica column followed by LH-20 column (Sephadex®) and crystallizing with a solvent at −5° C. to 0° C. to obtain a compound which is identified as Pterocarposide, represented by STR#1
i) Passing the enriched fraction 2 from step g) through isocratic RP-18 Silica column followed by LH-20 column (Sephadex®) column and crystallizing with a solvent at room temperature to obtain a compound which is identified as Sabioside, represented by STR#2.

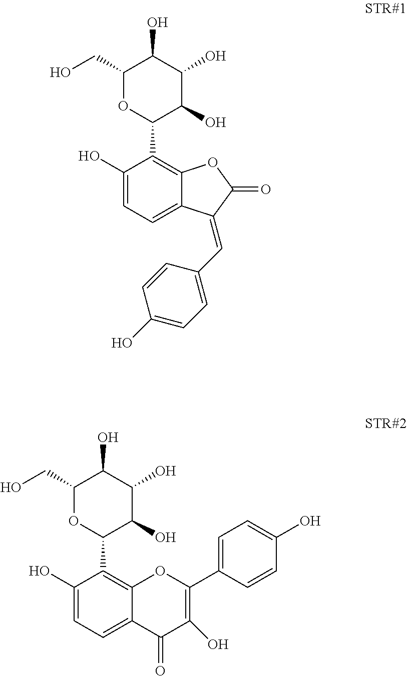 [SMILES: O=C1Oc2c(ccc(O)c2[C@@H]2O[C@H](CO)[C@@H](O)[C@H](O)[C@H]2O)/C1=C\c1ccc(O)cc1 ; O=c1c(O)c(-c2ccc(O)cc2)oc2c([C@@H]3O[C@H](CO)[C@@H](O)[C@H](O)[C@H]3O)c(O)ccc12]

In a related embodiment, the solvent is selected from the group consisting of, but not limited to methanol, ethanol, butanol, ethylacetate, chloroform, toluene, acetone and hexane.

In another preferred embodiment, the invention discloses a composition comprising not less than 5% w/w Pterocapus marsupium extract standardized to contain not less than 0.5% w/w Pterocarposide (STR#1) and not less than 0.5% w/w Sabioside (STR#2), said composition prepared using a process containing steps of:
a) Charging Pterocarpus marsupium wood powder into an extractor
b) Extracting with a solvent to obtain an oleoresin
c) Dissolving the oleoresin of step b) in water and extracting with a solvent to obtain a aqueous layer and solvent layer
d) Further washing the solvent layer of step c) with water to further obtain an aqueous and solvent fractions
e) Mixing the aqueous fractions of step c) and step d) and spray drying to obtain an aqueous extract
f) Fractionating the aqueous fraction of step e) with a solvent to obtain an aqueous fraction and solvent fraction
g) Passing the solvent fraction of step f) through a solvent gradient column by using Silica gel 60-120 mesh to obtain enriched fraction 1 and fraction 2
h) Passing the enriched fraction 1 from step g) through isocratic RP-18 Silica column followed by LH-20 column (Sephadex®) and crystallizing with a solvent at −5° C. to 0° C. to obtain a compound which is identified as Pterocarposide, represented by STR#1
i) Passing the enriched fraction 2 from step g) through isocratic RP-18 Silica column followed by LH-20 column (Sephadex®) column and crystallizing with a solvent at room temperature to obtain a compound which is identified as Sabioside, represented by STR#2.
j) Charging Pterocarpus marsupium aqueous extract of step e) into an extractor
k) Adding demineralized water to the extract and stirring for 3-4 hours at 65° C.-70° C. and leaving the solution idle for 8-10 hours for the insolubles to settle
l) Filtering the solution of step k) to remove the insolubles and obtain a clear filtrate
m) Checking the insolubles of step l) for the presence of Pterocarposide (STR#1) or Sabioside (STR#2), discarding if present in negligible amounts
n) Collecting the filtrate of step l) and extracting with a solvent twice to obtain a aqueous layer and solvent layer
o) Concentrating the solvent layer of step n) to recover the solvent
p) Extracting the aqueous layer of step n) with a solvent, thrice and combining the solvent fractions
q) Concentrating the solvent fractions and dissolving in water to standardize a solution containing 30% total dissolved solids
r) Spray drying the solution of step q) to obtain a composition comprising not less than 5% w/w Pterocarpus marsupium extract standardized to contain not less than 0.5% w/w Pterocarposide and not less than 0.5% w/w Sabioside, represented by STR#1 and STR#2 respectively.

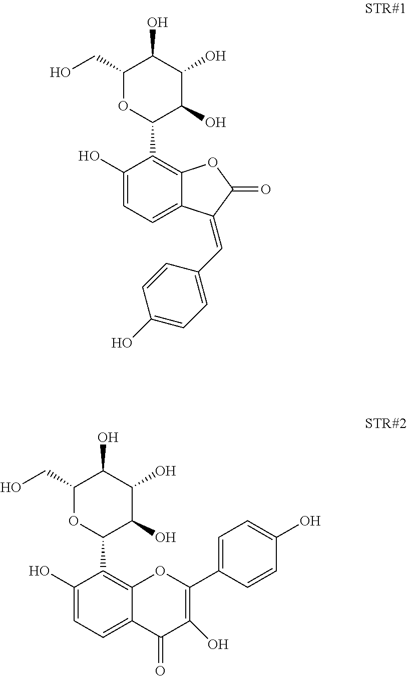 [SMILES: O=C1Oc2c(ccc(O)c2[C@@H]2O[C@H](CO)[C@@H](O)[C@H](O)[C@H]2O)/C1=C\c1ccc(O)cc1 ; O=c1c(O)c(-c2ccc(O)cc2)oc2c([C@@H]3O[C@H](CO)[C@@H](O)[C@H](O)[C@H]3O)c(O)ccc12]

In a related embodiment, the solvent is selected from the group consisting of, but not limited to methanol, ethanol, butanol, ethylacetate, chloroform, toluene, acetone and hexane.

In another preferred embodiment, the invention discloses a composition comprising not less than 5% w/w Pterocarpus marsupium extract standardized to contain not less than 0.5% w/w Pterocarposide (STR#1) and not less than 0.5% w/w Sabioside (STR#2).

In another preferred embodiment, the invention discloses a method of activating AMPK in mammalian cells, comprising step of bringing into contact mammalian cells with a composition comprising not less than 5% w/w Pterocarpus marsupium extract standardized to contain not less than 0.5% w/w Pterocarposide (STR#1) and not less than 0.5% w/w Sabioside (STR#2), to bring about the effect of AMPK activation. In a related embodiment, the mammalian cells are human cells.

In another preferred embodiment, the invention discloses a composition comprising not less than 5% w/w Pterocarpus marsupium extract standardized to contain not less than 0.5% w/w Pterocarposide (STR#1) and not less than 0.5% w/w Sabioside (STR#2), for use in activating AMPK in mammalian cells.

In another preferred embodiment, the invention discloses a method of inhibiting gluconeogenesis in mammalian cells, said method comprising steps of bringing into contact mammalian cells with a composition comprising not less than 5% w/w Pterocarpus marsupium extract standardized to contain not less than 0.5% w/w Pterocarposide (STR#1) and not less than 0.5% w/w Sabioside (STR#2), to bring about the effect of reduction in glucose production. In a related embodiment, the mammalian cells are human cells.

In another preferred embodiment, the invention discloses a composition comprising not less than 5% w/w Pterocarpus marsupium extract standardized to contain not less than 0.5% w/w Pterocarposide (STR#1) and not less than 0.5% w/w Sabioside (STR#2), for use in inhibiting gluconeogenesis in mammalian cells.

The following sections of this specification consist of illustrative examples of the most preferred embodiments of the present invention.

### EXAMPLES

### Example 1

### C-glycoside Composition and the Process for Preparing the Same

The C-glycosides from Pterocarpus marsupium are isolated and identified by the following steps:
a) Charging Pterocarpus marsupium wood powder into an extractor
b) Extracting with methanol to obtain an oleoresin
c) Dissolving the oleoresin of step b) in water and extracting with toluene to obtain a aqueous layer and toluene layer
d) Further washing the toluene layer of step c) with water to further obtain an aqueous and toluene fractions
e) Mixing the aqueous fractions of step c) and step d) and spray drying to obtain an aqueous extract
f) Fractionating the aqueous fraction of step e) with ethyl acetate to obtain an aqueous fraction and ethyl acetate fraction
g) Passing the ethyl acetate fraction of step f) through a solvent gradient column by using Silica gel 60-120 mesh to obtain enriched fraction 1 and fraction 2
h) Passing the enriched fraction 1 from step g) through isocratic RP-18 Silica column followed by LH-20 column (Sephadex®) and crystallizing with methanol at −5° C. to 0° C. to obtain a compound which is identified as Pterocarposide, represented by STR#1
i) Passing the enriched fraction 2 from step g) through isocratic RP-18 Silica column followed by LH-20 column (Sephadex®) column and crystallizing with acetone at room temperature to obtain a compound which is identified as Sabioside, represented by STR#2.

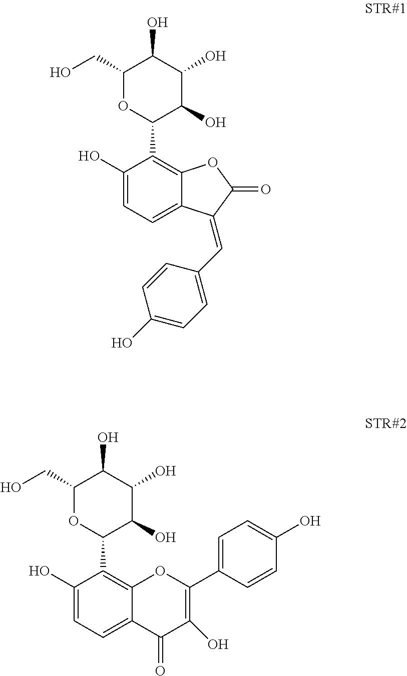 [SMILES: O=C1Oc2c(ccc(O)c2[C@@H]2O[C@H](CO)[C@@H](O)[C@H](O)[C@H]2O)/C1=C\c1ccc(O)cc1 ; O=c1c(O)c(-c2ccc(O)cc2)oc2c([C@@H]3O[C@H](CO)[C@@H](O)[C@H](O)[C@H]3O)c(O)ccc12]

The stereochemistry of the isolated Pterocarposide (STR#1) and Sabioside (STR#2) are provided herein below:

Pterocarposide
CAS no. 264876-26-8
Molecular formula: C₂₁H₂₀O₉
Chemical name: (3E)-7-β-D-glucopyranosyl-6-hydroxy-3-[(4-hydroxyphenyl)methylene]-2(3H)-Benzofuranone

Sabioside
CAS no. 108351-24-2
Molecular formula: C₂₁H₂₀O₁₀
Chemical name: 8-β-D-glucopyranosyl-3,7-dihydroxy-2-(4-hydroxyphenyl)-4H-1-Benzopyran-4-one

Further, a water soluble composition comprising not less than 5% w/w P. marsupim extract was prepared and was standardized to contain not less than 0.5% w/w Pterocarposide (STR#1) and not less than 0.5% w/w Sabioside (STR#2). The steps for preparing the composition are below:
a) Charging Pterocarpus marsupium aqueous extract into an extractor
b) Adding demineralized water to the extract and stirring for 3-4 hours at 65° C.-70° C. and leaving the solution idle for 8-10 hours for the insolubles to settle
c) Filtering the solution of step b) to remove the insolubles and obtain a clear filtrate
d) Checking the insolubles of step c) for the presence of Pterocarposide (STR#1) or Sabioside (STR#2), discarding if present in negligible amounts
e) Collecting the filtrate of step c) and extracting with a solvent twice to obtain a aqueous layer and solvent layer
f) Concentrating the solvent layer of step e) to recover the solvent
g) Extracting the aqueous layer of step e) with butanol, thrice and combining the butanol fractions
h) Concentrating the butanol fractions and dissolving in water to standardize a solution containing 30% total dissolved solids
i) Spray drying the solution of step h) to obtain a composition containing not less than 5% w/w of Pterocarposide and Sabioside, represented by STR#1 and STR#2 respectively.

### Example 2

### Activation of AMPK

Several experiments were conducted in H4IIE rat hepatoma cells and HepG2 human hepatoma cells. Confluent plates of H4IIE or HepG2 cells were treated with composition comprising Pterocarposide (STR#1) and Sabioside (STR#2) (Pterocarposide composition) or the positive control, 5-Aminoimidazole-4-carboxamide ribonucleotide (AICAR), at the following doses.

Pterocarposide composition doses: 0.05, 0.1, 0.5, and 1 μM

AICAR (positive control): 2 mM

Cells were lysed, and proteins were separated on 4-20% SDS-PAGE gel, and transferred to nitrocellulose. Activation of AMPK was detected by Western blotting with pAMPK (Thr172) and pACC (Ser79). AMPK and GAPDH were used as controls.

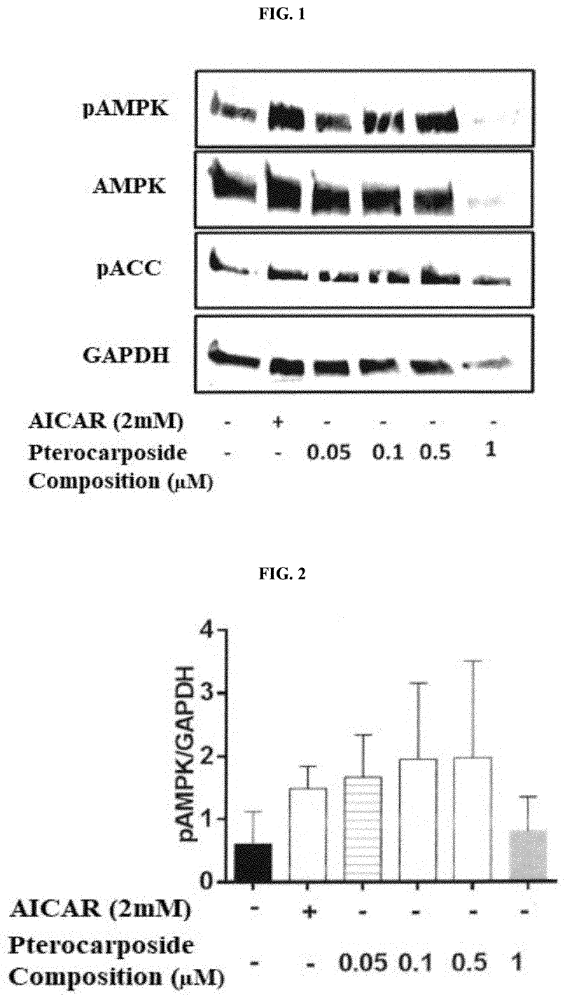

Results: The composition comprising Pterocarposide (STR#1) and Sabioside (STR#2) dose-dependently increased phosphorylation status of AMPK (Thr172), with maximal phosphorylation observed between 0.1-0.5 μM concentrations (FIG. 1). The composition also dose-dependently increased phosphorylation of acetyl CoA carboxylase (ACC). This is the first demonstration that the composition comprising Pterocarposide STR#1) and Sabioside (STR#2) activates AMPK. Increased ACC phosphorylation would indicate that the composition can inhibit fatty acid synthesis and potentially activate fatty acid oxidation. Similar findings were also observed in HepG2 cells (FIG. 2)

### Example 2

### Inhibition of Glucose Production

Confluent plates of H4IIE were treated with 0.1 μM or 0.5 μM of composition comprising Pterocarposide (STR#1) and Sabioside (STR#2) to examine its effects on dexamethasone-induced glucose production. H4IIE cells were treated with 500 nM dexamethasone and 0.1 mM 8-CTP-cAMP (Dex/cAMP}, various concentrations of Pterocarposide composition or 5 nM insulin in glucose production buffer (glucose-free DMEM medium, pH 7.4, containing 20 mM sodium lactate and 2 mM sodium pyruvate, without phenol red) for 5 hours.

Cells were washed with Dulbecco's PBS, and then incubated for 3 hours in glucose production buffer with the same concentrations of Dex/cAMP, insulin and Pterocarposide composition. Glucose production was assayed by measuring glucose concentration in the medium as described by Wang et al (2000) with modifications, using the glucose (HK) assay kit (Sigma Chemicals).

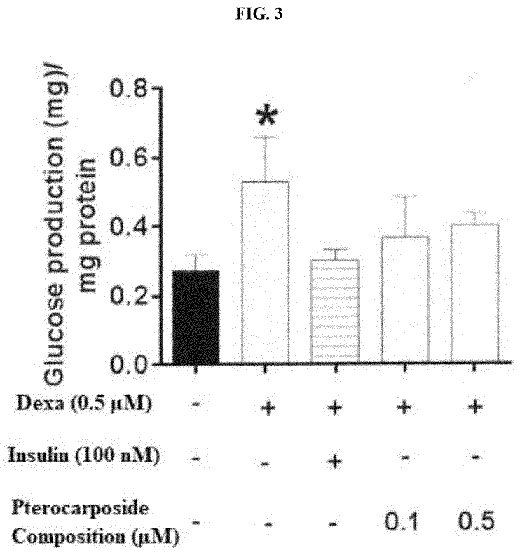

Results: Pterocarposide composition treatment (0.1 and 0.5 μM) inhibited dexamethasone-induced glucose production in H4IIE cells, to a similar extent as that of insulin (100 nM). Results are shown as mg of glucose produced±SEM (FIG. 3). Corrections for cell number were made on the basis of the protein concentration, assayed using Bio-Rad's Bradford protein assay reagent (Bio-Rad, Hercules, Calif.). (Wang, J. C., Stafford, J. M., Scott, D. K., Sutherland, C., Granner, D. K. (2000). The molecular physiology of hepatic nuclear factor 3 in the regulation of gluconeogenesis. The Journal of Biological Chemistry 275: 14717-14721)

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A composition comprising not less than 5% w/w Pterocarpus marsupium extract standardized to contain not less than 0.5% w/w pterocarposide (STR#1) and not less than 0.5% w/w sabioside (STR#2).

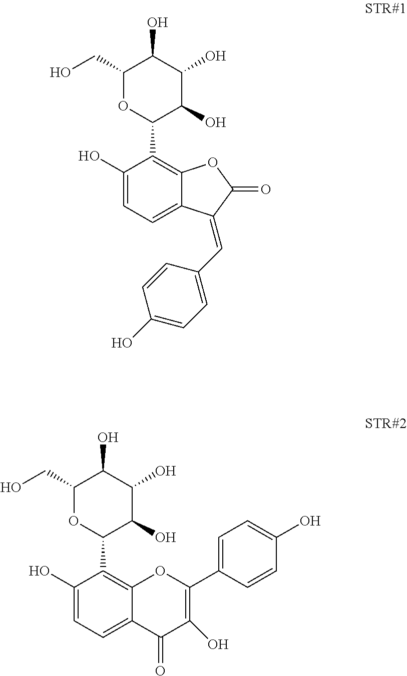 [SMILES: O=C1Oc2c(ccc(O)c2[C@@H]2O[C@H](CO)[C@@H](O)[C@H](O)[C@H]2O)/C1=C\c1ccc(O)cc1 ; O=c1c(O)c(-c2ccc(O)cc2)oc2c([C@@H]3O[C@H](CO)[C@@H](O)[C@H](O)[C@H]3O)c(O)ccc12]

2. The composition as in claim 1, wherein said composition is prepared using a process comprising steps of:
a) Charging Pterocarpus marsupium wood powder into an extractor
b) Extracting with a solvent to obtain an oleoresin
c) Dissolving the oleoresin of step b) in water and extracting with a solvent to obtain a aqueous layer and solvent layer
d) Further washing the solvent layer of step c) with water to further obtain an aqueous and solvent fractions
e) Mixing the aqueous fractions of step c) and step d) and spray drying to obtain an aqueous extract
f) Fractionating the aqueous fraction of step e) with a solvent to obtain an aqueous fraction and solvent fraction
g) Passing the solvent fraction of step f) through a solvent gradient column by using Silica gel 60-120 mesh to obtain enriched fraction 1 and fraction 2
h) Passing the enriched fraction 1 from step g) through isocratic RP-18 Silica column followed by LH-20 column (Sephadex®) and crystallizing with a solvent at −5° C. to 0° C. to obtain a compound which is identified as Pterocarposide, represented by STR#1
i) Passing the enriched fraction 2 from step g) through isocratic RP-18 Silica column followed by LH-20 column (Sephadex®) column and crystallizing with a solvent at room temperature to obtain a compound which is identified as Sabioside, represented by STR#2.
j) Charging Pterocarpus marsupium aqueous extract of step e) into an extractor
k) Adding demineralized water to the extract and stirring for 3-4 hours at 65° C.-70° C. and leaving the solution idle for 8-10 hours for the insolubles to settle
l) Filtering the solution of step k) to remove the insolubles and obtain a clear filtrate
m) Checking the insolubles of step l) for the presence of Pterocarposide (STR#1) or Sabioside (STR#2), discarding if present in negligible amounts
n) Collecting the filtrate of step l) and extracting with a solvent twice to obtain a aqueous layer and solvent layer
o) Concentrating the solvent layer of step n) to recover the solvent
p) Extracting the aqueous layer of step n) with a solvent, thrice and combining the solvent fractions
q) Concentrating the solvent fractions and dissolving in water to standardize a solution containing 30% total dissolved solids
r) Spray drying the solution of step q) to obtain a composition comprising not less than 5% w/w Pterocarpus marsupium extract standardized to contain not less than 0.5% w/w Pterocarposide and not less than 0.5% w/w Sabioside, represented by STR#1 and STR#2 respectively.

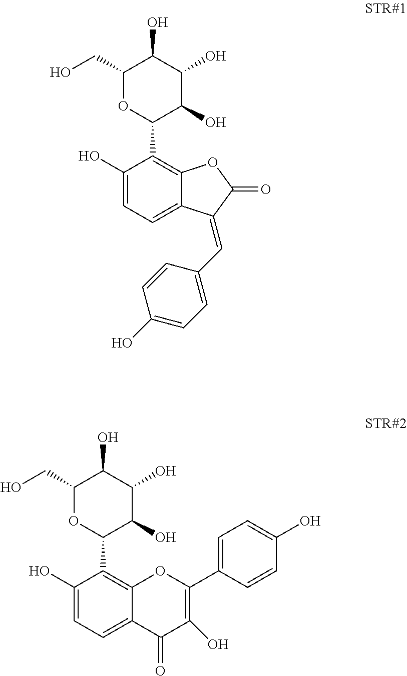 [SMILES: O=C1Oc2c(ccc(O)c2[C@@H]2O[C@H](CO)[C@@H](O)[C@H](O)[C@H]2O)/C1=C\c1ccc(O)cc1 ; O=c1c(O)c(-c2ccc(O)cc2)oc2c([C@@H]3O[C@H](CO)[C@@H](O)[C@H](O)[C@H]3O)c(O)ccc12]

3. The composition as in claim 2, wherein the solvent is selected from the group comprising of methanol, ethanol, butanol, ethylacetate, chloroform, toluene, acetone and hexane.

4. The composition as in claim 1 for use in activating AMPK in mammalian cells.

5. The composition as in claim 4, wherein the mammalian cells are human cells.

6. The composition as in claim 1 for use in inhibiting gluconeogenesis in mammalian cells.

7. The composition as in claim 6, wherein the mammalian cells are human cells.
